(19) United States
(12) Patent Application Publication
(10) Pub. No.: US 2025/0354949 A1
(43) Pub. Date: Nov. 20, 2025

(54) System for the Measurement of the Copper Percentage in White Metal in a Smelting Furnace

(71) Applicant: CODELCOTEC SPA, Región Metropolitana, Santiago (CL)

(72) Inventors: Leonel Contreras Rojas, Región Metropolitana, Santiago (CL); Víctor Duarte Olave, Región Metropolitana, Santiago (CL); Gonzalo Bravo, Región Metropolitana, Santiago (CL)

(21) Appl. No.: 17/756,584

(22) PCT Filed: Nov. 27, 2020
(86) PCT No.: PCT/CL2020/050165

§ 371 Date: May 27, 2022

(30) Foreign Application Priority Data
Nov. 29, 2019 (CN) 201911198609.3

(51) Int. Cl. G01N 27/07; C22B 9/00; F27D 21/00; G01N 33/202; G01N 33/2028

(52) U.S. Cl. CPC G01N 27/07; C22B 9/00; F27D 21/0028; G01N 33/202; G01N 33/2028; F27M 2003/13

(57) ABSTRACT

Provided is a system to measure the percentage of copper concentrate in the melting stage in-line and in real-time, it consists of at least four (1) electrodes inserted aligned through the refractory wall (2) of a smelting furnace, so that one end of each of the electrodes (1) remains on the outside of the furnace and the other end is inserted in the middle where the reaction occurs; i.e., inserted into the smelting bath, with these electrodes (1) connected to a signal amplifier which in turn is connected to signal generator, in which said power generator sends a replicated signal from the signal generator, sending the current-increased signals for charges with resistances of less than 0.1 ohm, and with bandwidths of 3 MHz, in which the power amplification sends the power signal to the electrodes (1) at the ends of the alignment so that the electrodes (1) that remain in the center receive the resistivity reading once the signal has been sent.

### TECHNICAL FIELD

This invention patent application is aimed at a system that measures the percentage of copper content of the main product of a melting furnace in-line and real-time, thus improving the efficiency of downstream processes. It is aimed specifically at a system that measures the copper concentrate in the melting stage and consists of aligned electrodes connected to a power amplifier. The electrodes are inserted into the melting furnace through the refractory wall or the cylinder head of the furnace, so that one end of each of the electrodes are on the outside of the furnace and the other end is inserted in the middle where the casting reaction takes place; i.e., insert in the casting bath and the resistivity they receive from those that take place inside the casting bath, thus being able to determine, based on its specific resistivity, the percentage of copper in the casting bath inside the furnace. The system allows the increase of the concentrate treatment capacity, the reduction of slag reprocessing and efficient use of the current asset, which results in a reduction of operational costs.

### BACKGROUND

The copper content is one of the most influential parameters in the quality of the white metal produced and the slag that is discarded during the melting stage. This value is planned by the casting according to its requirements, thus, planning can result in the furnace producing a white metal containing a certain percentage of Cu because it requires a greater amount of energy to consume a cold load in the subsequent conversion process, while in another planning, a higher Cu concentration may be required to generate a minimum level of slag.

Currently, in the melting process, there is a low level of instrumentation that implies not being able to obtain the quality of white metal required with a variability necessary for the operation. This is because no measurement is taken of the copper concentration in the white metal inside the furnace, either in-line or in real-time (according to the natural times of the process). On the contrary, this information is currently available with a 30- to 120-minute delay, which therefore delays the operators decisions. This is because a manual sample (jet tray, either in white metal or in slag) has to be taken. At present, there is no sensor to monitor the quality of the white metal inside the furnace on-line in order to extract it with the copper percentage required.

The publication of Patent JP2009068855 describes a device to measure copper concentrations in molten metal. The device consists of a probe that measures the copper concentration in iron casts, the methodology of which considers the measurement of copper activity. If consists of oxygen ion conductivity on the external surface of a solid electrolyte, onto which a secondary electrode that has copper oxides is placed is placed. A cover or top is also added that covers the electrolyte via the external part of the secondary electrode. Thus, in conjunction with this, an elemental device is structured for the use of normal electrodes coupled with an opposite electrode to establish a partial (or local) equilibrium between the copper oxides that is constituted by the secondary electrode, the oxygen and the copper inside the molten metal. In this manner, it focuses on depth to create the copper probe by an electrolyte that allows copper to be measured, which is different to the solution of this invention that focuses on the problem of electrochemical measurement of potentiometry.

The publication of Patent US2006250614 describes an analysis method and device for systems in molten phases using optical emission spectrometry, for example, in cast iron or steel, slag, glass, or lava. A sensitive element is used that has at least one emission spectrometer and at least one stimulus device. This is in order to excite the material to be analyzed and allow the total partial generation of radiation to be analyzed by the spectrometer present in the sensitive element. The aforementioned sensitive element comes into contact with the molten material that will be analyzed and transmits information, which contains analysis elements provided by a spectrometer. The invention also refers to an immersion sensor. Unlike this application, this publication is not considered to be a device with electrodes that measure by potentiometry.

The publication of patent US2003234928 describes a device and method that analyzes a material cast at high temperatures in real time using the LIBS Technique. This technique is applied to determine the elemental composition in solids, liquids and gases, and in summary, consists of exciting a sample by a luminous impulse. The expected response is the generation of plasma (ions, atoms, electrons) that are emitted in a radiation spectrum from the elements contained in the plasma.

The procedure uses a gas flow forced through a tube that can be inserted into the molten material in order to create a bubble. Then the inner surface of this bubble represents the molten material. In the case of an furnace or a converter, the laser impulse should be inserted perhaps by the nozzles (which, operationally would not be very feasible).

### SUMMARY

In view of the previous art mentioned above, there is the need to have a system to measure electrical conductivity through electrodes inside the bath. The electrolyte has a sensor that is capable of measuring the concentration of copper present in the bath, for example, white metal, inside a bath furnace, in such a manner that an operator has a tool to make timely decisions when to extract the liquid with the quality required.

According to an embodiment, a system to measure the percentage of copper concentrate in the melting stage in-line and in real-time is provided, which allows the increase of the treatment capacity of concentrates, the reduction of slag reprocessing and the efficient use of the circulating element, all of which translates into reduced operational costs, wherein because it consists of at least four electrodes inserted aligned through the refractory wall of a smelting furnace, so that one end of each of the electrodes remains on the outside of the furnace and the other end is inserted in the middle where the reaction occurs; i.e., inserted into the smelting bath, with these electrodes connected to a signal amplifier which in turn is connected to signal generator, in which said power generator sends a replicated signal from the signal generator, sending the current-increased signals for charges with resistances of less than 0.1 ohm, and with bandwidths of 3 MHZ, in which the power amplification sends the power signal to the electrodes at the ends of the alignment so that the electrodes that remain in the center receive the resistivity reading once the signal has been sent.

### BRIEF DESCRIPTION OF THE FIGURES

FIGS. 1 and 2: show an illustration of how the electrodes are installed on the refractory wall of a casting furnace.

FIGS. 3 and 4: show an image of the electrodes installed in the slag head of a Lieutenant converter.

FIGS. 5 and 6: show an image of the electrodes installed in the white metal cylinder head of a Lieutenant converter.

FIG. 7: shows a cross-section linear resistor A and L-length connected to a DC voltage source to explain Ohms law.

FIG. 8: shows a graph illustrating the electrical resistivity depending on the temperature for copper without considering volume correction.

FIG. 9: shows a graph of the volume expansion quotient based on the temperature for copper.

FIG. 10: shows a graph of electrical resistivity based on the temperature for copper, considering volume correction.

FIG. 11: shows a graph of electrical conductivity based on the temperature for copper without considering volume correction.

FIG. 12: shows a graph of electrical conductivity based on the temperature for copper, with volume correction.

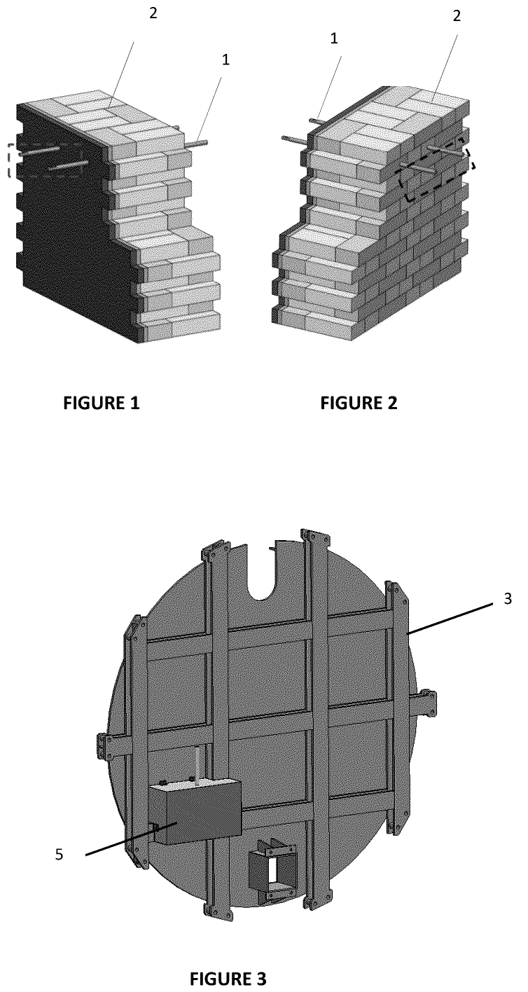

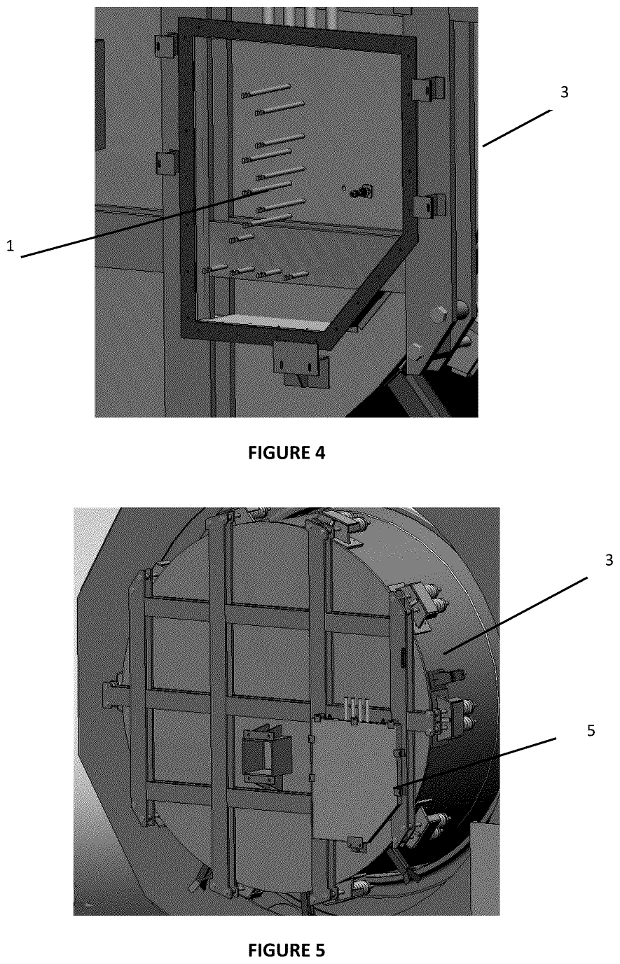

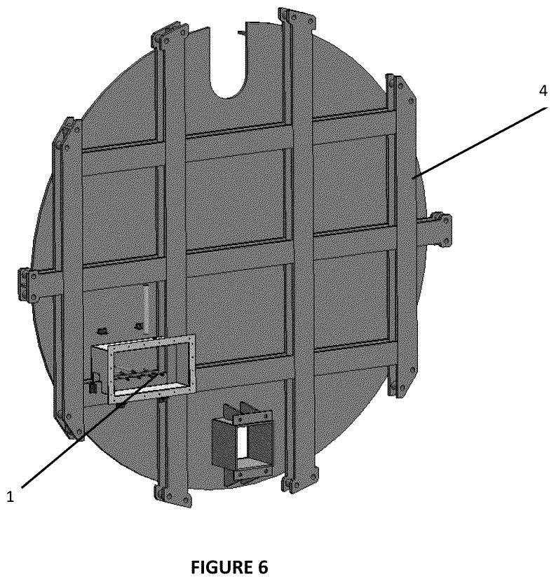

### DETAILED DESCRIPTION OF THE INVENTION

In order to measure the copper concentration continuously in liquid phases at temperatures greater than 1200 [° C.], the background of the general behavior of electrical conductivity and resistivity in terms of temperature is considered as a theoretical base.

In particular, depending on the experimental references, the differences in resistivity and electrical conductivity are obtained based on temperature. In terms of the theory, the conductivity of a material changes drastically when the phase changes because the charge transportation mechanism and its interaction with the constituent particles of the material changes its nature. In the case of a solid material, the theory refers to “phonons”, which are arrangements of energy that travel through the crystalline network and interact with the charge carriers, obstructing their path, implying a change of conductivity at macroscopic level. In the case of liquid phases, the model refers to ionic solutions, in which the electrical charges interact with each other by Coulomb force, where the temperature randomly influences the velocity of the particles present in the solution. For example, electrical conductivity in a solid-state material is of the order of 9×10⁷ [1/Ohm×m], whereas in a liquid state, it is 4×10⁷ [1/Ohm×m].

The electromagnetic constituent parameters of a material medium are its electrical permittivity ε, magnetic permeability μ and conductivity σ. It is said that a material is homogeneous or uniform if its constitutive parameters do not vary from one point to another and in turn is isotropic, if its constitutive parameters are independent from the direction. There are a lot of materials that exhibit isotropic properties; however, not all crystalline solids, or liquids for that matter, possess this distinctive feature.

Electrical conductivity easily measures how electrons can travel through the material influenced by an external electric field. Materials are classified as electric conductors (metals) or dielectric (insulating) depending on the magnitudes of their conductivity.

A conductor has a large number of electrons adhered weakly to the outermost layers of atoms. Without an external electrical field, these free electrons move in random directions at variable speeds. Its random movement produces a zero average current through the conductor. However, when applying an external electrical field, the electrons migrate from one atom to the next along a direction contrary to that of the external field. Its movement, which is characterized by an average velocity, known as electron flow velocity, causes a conduction current.

In a dielectric material, the electrons are strongly adhered to the atoms, which implies that it is difficult to detach them under the application of an electric field. Consequently, no current flows through the material.

A perfect dielectric is a material with a σ of almost zero, and in contrast, a perfect conductor is a material with a very large σ. The electrical conductivity of most metals is within the range of 10⁶ to 10⁷ [1/Ohm×m], compared to 10⁻¹⁰ to 10⁻⁷ of good insulators (See Table 1).

TABLE 1
Electrical Conductivity of some common
materials at 20° C. (and low frequency)

| Material | Conductivity σ [1/ohm × m] |
|---|---|
| Conductors | |
| Silver | 6.2 × 10⁷ |
| Copper | 5.8 × 10⁷ |
| Gold | 4.1 × 10⁷ |
| Aluminum | 3.5 × 10⁷ |
| Iron | 10⁷ |
| Mercury | 10⁶ |
| Coal | 3 × 10⁴ |
| Semiconductor | |
| Pure Germanium | 2.2 |
| Pure Silicon | 4.4 × 10⁻⁴ |
| Insulators | |
| Glass | 10⁻¹² |
| Paraffin | 10⁻¹⁵ |
| Mica | 10⁻¹⁵ |
| Fused Quartz | 10⁻¹⁷ |

The materials with electrical conductivity between that of conductors and insulators are known as semiconductors.

The electrical conductivity of a material depends on a number of factors, including temperature and the presence of impurities. In general in metals, conductivity decreases with the increase of the temperature and on the other hand, at very low temperatures close absolute zero, some conductors become superconductors because their conductivity levels become very high.

A perfect conductor is an equipotential medium, which means that the electrical potential is the same at all points of the conductor. This property is derived from that the difference between two points of the conductor is equal by definition, equal to the total of the field line between two points. However, the field is equal to zero in all parts of the conductor, so the voltage difference is zero. However, the fact that the conductor is an equipotential medium does not necessarily imply that the difference of potential between the conductor any other conductor is zero. Each conductor is an equipotential medium, but the presence of different charge distributions on its surfaces can generate a difference in potential between them.

The Ohm [symbol] law is used on this occasion to introduce another important term that is more usable in references: resistivity:

$$\vec{J} = σ \vec{E} \qquad ( 1 )$$

\vec{J}: Current density vector,
σ: Electrical Conductivity.
\vec{E}: Electric field vector.

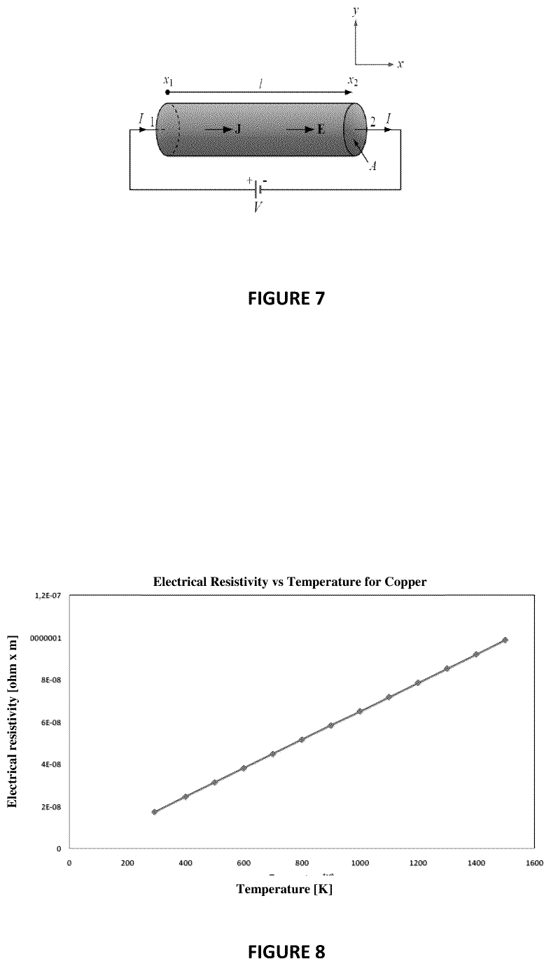

FIG. 7 shows a conductor length L, resistance R, and cross-section A. The conductor [symbol] axis is oriented along the x-axis and extends between the X-Points₁ and X₂, with L=X₂−X₁.

A voltage V applied between the terminals of the conductor establishes an electric field:

$$\vec{E} = Ex \hat{i}$$

The E-direction is the highest potential point (point 1 in FIG. 7) to the lowest potential point (point 2 FIG. 7).

The relationship between the voltage and the electrical field component in X is obtained as follows:

$$V = {V}_{2} - {V}_{1} = - {\int }_{{x}_{2}}^{{x}_{1}} \vec{E} \cdot d \vec{l} = {E}_{x} l \qquad ( 2 )$$

The current flowing through section A of the conductor is the total of the current density on the surface:

$$I = {\int }_{A} \vec{J} \cdot d \vec{S} = {\int }_{A} σ \cdot \vec{E} \cdot d \vec{S} = σ {E}_{x} \overline{A} \qquad ( 3 )$$

On the other hand, the best known relationship of Ohm [symbol] Law and (2) and (3) is:

$$R = \frac{l}{σ A} = ρ \frac{l}{A} \qquad ( 4 )$$

Where ρ is another important parameter, known as electrical resistivity, and is the inverse of conductivity. The latter is the most studied in temperature-based behavior. In this manner, by establishing its shape, the electrical conductivity be indirectly established.

According to the theories, electrical resistivity and therefore the resistance in a conductor depend on the temperature, and in many cases, it can be assumed that resistivity depends linearly.

In a temperature range not too large, the resistivity of a metal can be represented approximately by the following equation:

$${ρ}_{T} = {ρ}_{0} \cdot [ 1 + α \cdot ( T - {T}_{0} ) ] \qquad ( 5 )$$

Where ρ₀ is the resistivity at a reference temperature T₀ (usually taken as at 20° C. or the ambient temperature) and ρ_{T} resistivity at a temperature T. The factor α is known as the resistivity temperature coefficient. Table 2 show the representative values of this coefficient.

TABLE 2
Resistivity temperature coefficients.

| Material | α[° C.⁻¹] |
|---|---|
| Aluminum | 0.0039 |
| Carbon | −0.0005 |
| Copper (recognized commercial) | 0.00393 |
| Constantan (Cu 60, Ni 40) | 0.000002 |
| Iron | 0.0050 |
| Brass | 0.0020 |
| Manganin (Cu 84, Mn 12, Ni 4) | 0.000000 |
| Mercury | 0.00088 |
| Nichrome | 0.0004 |
| Silver | 0.0038 |
| Lead | 0.0043 |
| Tungsten | 0.0045 |

It may be the case that electrical resistivity varies non-linearly with temperature, which implies that it is advisable to express this property in terms of potency series:

$$ρ = {ρ}_{0} ( 1 + {\sum }_{i} {{α}_{i} ( Δ T )}^{i} ) \qquad ( 6 )$$

Of all the known references and also considering that copper is a metal, electrical resistivity has an increasing linear behavior with temperature. In addition, when it reaches its melting point, its electrical resistivity increases, particularly for copper, in the increase is approximately double, as basically the volume changes inside the metal.

For example, FIG. 8 shows the electrical resistivity graph for copper based on temperature, without correction for volumetric change, using the equation (5) with T₀=293 K and ρ₀=1.72×10⁻⁸ [Ohm×m].

The expansion of volume is known to derive from thermodynamics depending on the change in length, as an extension of the linear expansion of solids subjected to temperature (in classical thermodynamics):

$$\frac{Δ V}{{V}_{0}} = 3 \cdot ( \frac{Δ L}{{l}_{0}} ) + 3 {( \frac{Δ L}{{l}_{0}} )}^{2} + {( \frac{Δ L}{{l}_{0}} )}^{3} \qquad ( 7 )$$

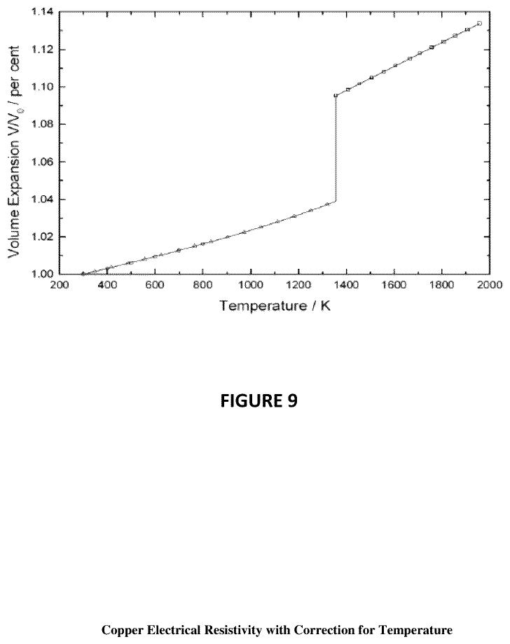

Then the graph of volume ratio variation based on temperature can be obtained, as observed in FIG. 9. From this graph, it can be observed that at the melting point≈1356 [K] or 1083 [° C.], there is a change in the quotient from 1.037 to 1.095. Since electrical resistivity depends implicitly on length variables, it is also affected by the change in volume. In practice, this means that the current must be extended in a greater space; therefore, the “body or surface” becomes more resistive.

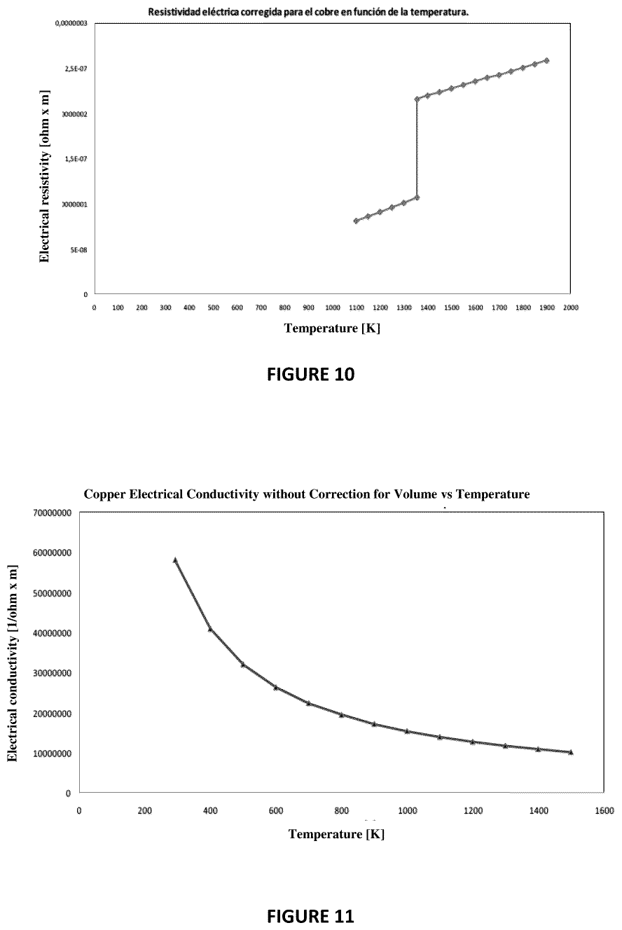

By making the respective transformations, the graph in FIG. 10 that illustrates the resistivity corrected according to the temperature with the following equations that parameterize the functions:

$${ρ}_{corr} ( T ) = - 0.02861 + 9.98873 \times {10}^{- 5} \begin{matrix}T & 1100 K < T < 1356 K\end{matrix} \\ {ρ}_{corr} ( T ) = 0.11031 + 7.83066 \times {10}^{- 5} \begin{matrix}T & 1356 K < T < 1900 K\end{matrix}$$

Based on the foregoing, it is known that electrical resistivity is the “multiplicative inverse” of electrical conductivity, and therefore, in practice the behavior of electrical conductivity is:

$${σ}_{T} = \frac{1}{{ρ}_{0} \cdot [ 1 + α \cdot ( T - {T}_{0} ) ]} \qquad ( 8 )$$

Based on temperature, the graph of this equation 8 would be as shown in FIG. 11. According to the foregoing, the correction of the volume in the transformation from solid to liquid state modifies the electrical resistivity values depending on the temperature (since they do not modify the form), implies that given the increase in volume, a fluid is more resistant to the passage of current. On the contrary, the electrical conductivity has an inverse behavior: the greater the volume of the body or bath to be investigated, it is less conductive; therefore, the decrease is greater due to the high temperatures.

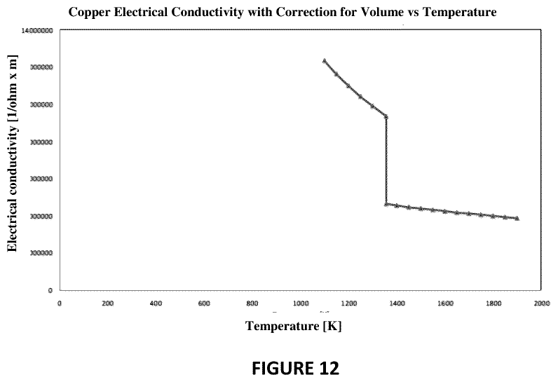

As can be seen in the graph in FIG. 12, the value of the electrical conductivity of copper passing the melting point decreases from 9×10⁷ to 4×10⁷ [1/Ohm×m] approximately, which implies that, in practice, the values should not be greater than 9×10⁷ (values assignable to the solid state).

Considering the aforementioned concepts, the system used to measure the percentage of copper in a casting bath considers that the higher the copper concentrations are in a molten metal bath (e.g., white metal), its electrical conductivity is greater. In practice, this should be consistent insofar that if a white metal sample with a 72.8% copper content should show a conductivity below 73.8.

From the point of view of measurement, the system allows frequencies to be varied frequencies and samples of white metal to be compared with different copper contents in a molten state its alternating current electrical conductivity to be measured using 4-wire sensing (Kelvin) techniques. This technique eliminates the need for wiring and contact potentials. It is very useful to measure very low-value resistances using special geophysical prospecting applications. The technique was originally developed by Lord Kelvin, later perfected by Frank Wenner at the beginning of the 20th century, who used it to measure the resistivity of soil samples. In geophysics, this technique is known as Wenner Method. It is most common to measure a resistance of intermediate values (tenths of [Ohm [symbol] ] to a few mOhm) with two points using a multimeter.

Thus, the system of the invention consists of at least four aligned electrodes (1) that are inserted through the refractory wall (2) of a smelting furnace, so that one end of each of the electrodes (1) remains on the outside of the furnace and the other end is inserted in the middle where the casting reaction occurs; i.e., inserted in the smelting bath.

The electrodes (1) are connected to a signal amplifier, which is an amplifier that receives a signal from a signal generator connected to the amplifier and sends them to the electrodes (1). Strictly speaking, the amplified signal consists of sending a low-voltage current, in the order of 6 V, but with a high current, in the order of the 30 A, in such a manner so as to interfere as little as possible with the resistivity of the liquid copper in the bath and thus measure the changes observed as its state changes during the reaction process.

Specifically, the amplified signal consists of a signal increased in current for charges with resistances below 0.1 ohm, and with bandwidths of 3 MHz, in which the power amplification sends the power signal to the electrodes (1) arranged on the ends of the alignment, so that the electrodes (1) arranged in the center receive the resistivity reading once the signal has been sent. To do so, the electrodes arranged in the center of the alignment are connected to a data processor that interprets the resistivity reading of these electrodes as a percentage of copper present in the smelting bath in the melting furnace.

In a preferred mode of execution, the electrodes (1) are inserted aligned through the slag head wall (3) of a melting converter and in another preferred mode the electrodes (1) are inserted aligned through the head wall of white metal (4) of a melting converter. In both cases, the electrodes (1) are covered by an enclosure on the outside of the converter (5).

The electrodes (1) are formed or steel refractory bars, which have conditions suitable for balancing resistivity. The resistance increase of steel refractory bars is very slow due to aging: they can increase with a ratio of approximately 5-6% for every 1,000 hours of continuous operation at 1,400° C. and a ratio of 3% for every 1,000 hours of continuous operation at 1,000° C.

1. System to measure the percentage of copper concentrate in the melting stage in-line and in real-time, which allows the increase of the treatment capacity of concentrates, the reduction of slag reprocessing and the efficient use of the circulating element, all of which translates into reduced operational costs, CHARACTERIZED because it consists of at least four (1) electrodes inserted aligned through the refractory wall (2) of a smelting furnace, so that one end of each of the electrodes (1) remains on the outside of the furnace and the other end is inserted in the middle where the reaction occurs; i.e., inserted into the smelting bath, with these electrodes (1) connected to a signal amplifier which in turn is connected to signal generator, in which said power generator sends a replicated signal from the signal generator, sending the current-increased signals for charges with resistances of less than 0.1 ohm, and with bandwidths of 3 MHz, in which the power amplification sends the power signal to the electrodes (1) at the ends of the alignment so that the electrodes (1) that remain in the center receive the resistivity reading once the signal has been sent.

2. System to measure the percentage of copper concentrate in the melting stage in-line and real-time, in accordance with claim 1 is CHARACTERIZED because said electrodes are formed of steel refractory bars.

3. System to measure the percentage of copper concentrate in the melting stage in-line and real-time, in accordance with claim 1 is CHARACTERIZED because the signal generator is a device that generated different signal patterns that allow the behavior of the molten material to be analyzed, depending on the responses of the signals measured, both in terms of amplitude, current, lag, frequency runs, quadrature, resonance, attenuation and/or voltage increase over time.

4. System to measure the percentage of copper concentrate in the melting stage in-line and real-time, in accordance with claim 1 is CHARACTERIZED because it is also understood that these electrodes (1) are inserted aligned through the wall of the slag head (3) of a melting converter.

5. System to measure the percentage of copper concentrate in the melting stage in-line and real-time, in accordance with claim 1 is CHARACTERIZED because it is also understood that these electrodes (1) are inserted aligned through the wall of the white metal head (4) of a melting converter.

6. System to measure the percentage of copper concentrate in the melting stage in-line and real-time, in accordance with claims 1, 4 and 5 is CHARACTERIZED because the electrodes (1) are enclosed on the outside of the converter (5).

7. System to measure the percentage of copper concentrate in the melting stage in-line and real-time, in accordance with claim 1 is CHARACTERIZED because the electrodes that remain in the center of the alignment are connected to a data processor that interprets the resistivity reading measured by these electrodes as a percentage of copper present in the smelting bath in the melting furnace.
